(19) United States
(12) Patent Application Publication
(10) Pub. No.: US 2022/0270716 A1
(43) Pub. Date: Aug. 25, 2022

(54) CONFIDENCE EVALUATION TO MEASURE TRUST IN BEHAVIORAL HEALTH SURVEY RESULTS

(71) Applicant: Ellipsis Health, Inc., San Francisco, CA (US)

(72) Inventors: Elizabeth Shriberg, Berkeley, CA (US); Yang Lu, Waterloo (CA); Amir Harati, Toronto (CA)

(21) Appl. No.: 17/493,687

(22) Filed: Oct. 4, 2021

Related U.S. Application Data
(63) Continuation of application No. PCT/US2020/026459, filed on Apr. 2, 2020.
Application No. PCT/US2020/026459 is a continuation-in-part of application No. 16/377,090, filed on Apr. 5, 2019, now abandoned.

(51) Int. Cl. G16H 10/20

(52) U.S. Cl. CPC G16H 10/20

(57) ABSTRACT

The present disclosure provides systems, methods, and computer program products for assessing the reliability of health survey results. An example can comprise: (a) determining that each of a predetermined pair of events are present in response data generated by a subject in response to prompts presented to the subject in administration of a health survey, wherein the pair of events includes a conditioning event and a conditioned event; (b) determining a probability that the conditioned event is present in the response data given the presence of the conditioning event in the response data; (c) repeating steps (a) and (b) for each of two or more predetermined pair of events; and (d) combining the probabilities to form a confidence vector data that represents a measure of confidence in the reliability of the subject in generating the response data.

### CROSS-REFERENCE

This application is a continuation application of International Application No. PCT/US 2020/026459, filed on Apr. 2, 2020, which application is a Continuation-In-Part of U.S. patent application Ser. No. 16/377,090, filed on Apr. 5, 2019, which applications are incorporated herein by reference in their entirety.

### BACKGROUND

Behavioral health is a serious problem. The most widely used tools for screening for behavioral health conditions may rely on accurate self-reporting by the screened public. For example, the current “gold standard” for questionnaire-based screening for depression is the Patient Health Questionnaire-9 (PHQ-9), a written depression health survey with nine multiple-choice questions. Other similar health surveys include the Patient Health Questionnaire-2 (PHQ-2) and the Generalized Anxiety Disorder 7 (GAD-7).

### SUMMARY

Behavioral health survey results may be inaccurate or otherwise unreliable. Response may be unreliable for a number of reasons, e.g., boredom, inattention, or distraction of the survey taker while taking the test; cheating by answering questions in a way believed to yield a particular result; or lack of ability to answer the questions due to lack of language proficiency, illiteracy, and lack of understanding of the question(s).

Such unreliable responses can lead to misdiagnoses of survey takers. However, consequences of unreliable responses can extend far beyond the correctness of a diagnosis of a given survey taker. Unreliable responses can render any statistical analysis or modeling of the corpus less accurate and less useful. Examples include analysis for population assessments, for monitoring, or for assessment of therapeutic treatments including medications. Examples also include AI systems that are trained to predict depression and that use the survey data as ground truth estimates for model training and evaluation. Some percentage of the survey data used for analysis, interpretation or machine learning based models will contain problems of the types just mentioned, resulting in suboptimal interpretations and suboptimal models.

What is needed is a way to identify which survey results may be affected by lack of reliability for the reasons above, so that end users of the surveys can decide whether or not to include the surveys for their purposes. Instead of a simple binary yes/no guess at which surveys are not to be trusted, what is needed is a score, or “confidence” to represent the estimated veracity or reliability of the particular survey data. End users can then threshold the scores based on the tolerance for corruption risk in their survey data, for their particular application. in survey taker responses in health survey analysis tools.

The present disclosure provides systems, methods, and computer program products for assessing the reliability of responses in a behavioral health survey. The systems described herein can process behavioral health survey results and output a score that is monotonically related to the estimated veracity of the results. The degree of confidence represented by the score may reflect testing for multiple types of consistencies. Such consistencies may include, but are not limited to, consistencies of the survey answer patterns with respect to a set of prior survey data, and conditional consistencies based on characteristics of the survey taker and the survey context. The systems can also implement a process in which a survey taker takes the same survey more than once and the system then computes additional reliability measures using consistencies across corresponding questions of the multiple surveys. In addition, the system can output real-time estimates that can be used to intervene in the survey administration process, resulting in better quality and/or cost savings for both the survey taker and the survey administration team.

Given these confidence annotations, any analysis of behavioral health survey results, e.g., statistical analysis and computational modeling through artificial intelligence (AI) such as deep machine learning, can be significantly more accurate and useful. For example, in such analysis, survey results with lower confidence can be weighted less or disregarded altogether while survey results with higher confidence can be weighted more heavily.

Identifying health survey results with low confidence may provide a number of significant advantages. For example, health survey results with low confidence may be culled from a corpus of health survey results. This may improve the results of any analysis performed on the corpus as a whole, including statistical analysis and artificial intelligence (AI) analysis. Any modeling of such a corpus of health survey results may yield much better results.

Another significant advantage is that survey takers whose health survey results are consistently unreliable can be identified. The health survey results of such survey takers can be the result of inattentiveness, intent to influence the survey results and indications, illiteracy, or insufficient proficiency in the language of the health survey, for example. Collecting a subset of the corpus of health survey results by inconsistently reliable survey takers can enable analysis and modeling to identify such survey takers early and to improve health surveys to obtain more accurate and reliable results for such survey takers.

In one aspect, the present disclosure provides a method for measuring a degree of confidence in a reliability of responses received from a human subject in a health survey for evaluating a health state of the subject. The method can comprise obtaining response data that is generated by the subject in response to prompts presented to the subject during administration of the health survey to the subject. The response data can comprise a plurality of conditioning events and a plurality of conditioned events. The method can further comprise determining a first probability that a first conditioned event is present in the response data based in part on a presence of a first conditioning event in the response data. The plurality of conditioning events can comprise the first conditioning event and the plurality of conditioned events can comprise the first conditioned event, and the first probability can be used to determine a first event pair comprising the first conditioned event and the first conditioning event. The method can further comprise repeating steps (a) and (b) for two or more other conditioned events and other conditioning events to generate a plurality of additional probabilities for a plurality of additional event pairs. The method can further comprise combining two or more probabilities selected from (i) the first probability and the plurality of additional probabilities or (ii) the plurality of additional probabilities to generate a confidence vector data. The confidence vector data can represent a measure of confidence in the reliability of the subject that generated the response data in response to the health survey.

In another aspect, the present disclosure provides a method for measuring a degree of confidence in a reliability of responses received from a human subject in a health survey for evaluating a health state of the subject. The method can comprise (a) administering the health survey to the subject to cause the subject to generate first response data in response to one or more constituent prompts of the health survey; (b) after administering the health survey, prompting the subject to perform an activity that is unrelated to the health survey; (c) after prompting the subject to perform the activity that is unrelated to the health survey, administering the health survey to the subject again to cause the subject to generate second response data in response to the one or more constituent prompts of the health survey; and (d) measuring one or more differences between the first response data and the second response data regarding corresponding the one or more constituent prompts of the health survey, in order to generate confidence data representing a degree of confidence in the reliability of the responses received from the subject in the health survey.

In some embodiments, measuring the one or more differences comprises: calculating absolute differences between responses in the first response data and the second response data corresponding to a same prompt of the health survey; determining a number of prompts of the health survey for which the absolute difference between responses in the first and second response data are at least a predetermined minimum threshold; and generating the confidence data based in part on the number of prompts.

In some embodiments, measuring the one or more differences further comprises: statistically normalizing the number of prompts to form a normalized number; and generating the confidence data based in part on the normalized number.

In some embodiments, measuring the one or more differences comprises: calculating absolute differences between responses in the first response data and the second response data corresponding to a same prompt of the health survey; determining a greatest absolute difference from among the absolute differences; and generating the confidence data based in part on the greatest absolute difference.

In some embodiments, measuring the one or more differences further comprises: statistically normalizing the greatest absolute difference to form a normalized greatest absolute difference; and generating the confidence data based in part on the normalized greatest absolute difference.

In some embodiments, measuring the one or more differences comprises: calculating absolute differences between responses in the first response data and the second response data corresponding to a same prompt of the health survey; determining a sum of the absolute differences; and generating the confidence data based in part on the sum of the absolute differences.

In some embodiments, measuring the one or more differences further comprises: statistically normalizing the sum of the absolute differences to form a normalized sum; and generating the confidence data based in part on the normalized sum.

In another aspect, the present disclosure provides a method for measuring a degree of confidence in a reliability of responses received from a human subject in a health survey for evaluating a health state of the subject. The method may comprise: (a) receiving response data generated by the subject in response to a plurality of prompts presented to the subject during administration of the health survey to the subject; (b) receiving health survey metadata data related to the administration of the health survey to the subject, the health survey metadata including data representing at least one item of information comprising information about the subject and/or information about an activity of the subject during the administration of the health survey; (c) determining a plurality of probabilities that the subject had generated the response data with reliability based in part on a presence of two or more items of information represented in the health survey metadata; and (d) combining the plurality of probabilities for different items of information represented in the health survey metadata to generate a confidence vector data. The confidence vector data may represents a measure of confidence in the reliability of the subject that generated the response data in response to the health survey.

In another aspect, the present disclosure provides a method for measuring a degree of confidence in a reliability of responses received from a human subject in a health survey for evaluating a health state of the subject. The method may comprise: (a) obtaining first response data that is generated by the subject in response to prompts presented to the subject during a first administration of the health survey to the subject, wherein the first response data comprises a plurality of conditioning events and a plurality of conditioned events; (b) determining a first probability that a conditioned event is present in the first response data based on a presence of a first conditioning event in the first response data, wherein the plurality of conditioning events comprises the first conditioning event and the plurality of conditioned events comprises the first conditioned event, and wherein the first probability is used to determine a first event pair comprising the first conditioned event and the first conditioning event; (c) repeating steps (a) and (b) for two or more other conditioned events and other conditioning events to generate a plurality of additional probabilities for a plurality of additional event pairs; (d) combining two or more probabilities selected from (i) the first probability and the plurality of additional probabilities or (ii) the plurality of additional probabilities to generate a first confidence vector data that represents a first measure of confidence in the reliability of the subject that generated the first response data in response to the health survey; (e) receiving health survey metadata data related to the administration of the health survey to the subject, the health survey metadata including data representing at least one item of information comprising information about the subject and/or information about an activity of the subject during the administration of the health survey; (f) determining a set of probabilities that the subject had generated the first response data with reliability based in part on a presence of two or more items of information represented in the health survey metadata; (g) combining the set of probabilities for different items of information represented in the health survey metadata to generate second confidence vector data that represents a second measure of confidence in the reliability of the subject that generated the first response data in response to the health survey; and (h) combining the first confidence vector data and the second confidence vector data to form an aggregate confidence vector data that represents an aggregate measure of confidence in the reliability of the subject that generated the first response data in response to the health survey.

In some embodiments, the method further comprises: (i) after receiving the first response data from (a), prompting the subject to perform an activity that is unrelated to the health survey; (j) administering the health survey to the subject a second time to obtain second response data in response to the prompts of the health survey; (k) determining differences between the first response data and second response data to generate third confidence vector data that represents a third measure of confidence in the reliability of the subject in generating the response data; and wherein step (h) comprises combining the first, second, and third confidence vector data to form aggregate confidence vector data that represents an aggregate measure of confidence in the reliability of the subject in generating the first and second response data.

In some embodiments, determining differences comprises: calculating absolute differences between responses in the first and second response data corresponding to the same prompt of the health survey; determining a number of prompts of the health survey for which the absolute difference between responses in the first and second response data are at least a predetermined minimum threshold; and generating the confidence data based at least in part on the number of prompts.

In some embodiments, determining differences further comprises: statistically normalizing the number of prompts to generate a normalized number; and generating the confidence data based at least in part on the normalized number.

In some embodiments, determining differences comprises: calculating absolute differences between responses in the first and second response data corresponding to the same prompt of the health survey; determining a largest one of the absolute differences; and generating the confidence data based at least in part on the greatest absolute difference.

In some embodiments, determining differences further comprises: statistically normalizing the greatest absolute difference to generate a normalized greatest absolute difference; and generating the confidence data based at least in part on the normalized greatest absolute difference.

In some embodiments, determining differences comprises: calculating absolute differences between responses in the first and second response data corresponding to the same prompt of the health survey; determining a sum of the absolute differences; and generating the confidence data based at least in part on the sum of the absolute differences.

In some embodiments, determining differences further comprises: statistically normalizing the sum of the absolute differences to generate a normalized sum; and generating the confidence data based at least in part on the normalized sum.

In another aspect, the present disclosure provides a method for improving an accuracy of a corpus of data comprising responses by two or more human subject in taking two or more health surveys that screen for a health state. The method can comprise (a) identifying two or more features of the corpus that includes human subject data that represents two or more human subjects and, for each of the subjects, response data that represents response data generated by the subject in response to prompts presented to the subject in administration of the health survey, wherein each of the features represents a data field within the human subject data or the response data; (b) for each combination of the two or more features, calculating mutual information of the combination within the corpus; (c) combining the mutual information of the combinations to generate an aggregate mutual information measure of the corpus; and (d) removing weak subject data or record data from the corpus, wherein weak subject data or record data includes subject data or record data whose removal from the corpus most increases the aggregate mutual information measure of the corpus.

In some embodiments, combining the mutual information comprises: calculating a weighted mean of the mutual information of the combinations.

In some embodiments, the method further comprises: repeating steps (a)-(d) if the removing in (d) does not reduce the size of the corpus below a predetermined minimum corpus size.

In some embodiments, the method further comprises repeating (a)-(d) until the aggregate mutual information measure of the corpus is at least a predetermined threshold.

In another aspect, the present disclosure provides a method for improving the quality of a behavioral health survey administered to a human subject using a client device. The method can comprise sending prompt data to the client device so as to cause the client device to present at least one but fewer than all of two or more prompts of the behavioral health survey to the subject; receiving response data from the client device, wherein the response data represents corresponding responses of the subject to each of the at least one presented prompts; measuring a degree of confidence in the reliability of responses represented by the response data; determining that the degree of confidence is below a predetermined threshold; in response to the determining, intervening in the survey by changing a manner in which the remainder of the survey is to be conducted through the client device.

In some embodiments, intervening comprises sending intervention data to the client device wherein the intervention data causes the client device to present a message to the subject to influence the subject's responses to prompts other than the presented prompts. In some embodiments, the intervention data causes the client device to present at least one of the presented prompts to the subject a second time and send second response data representing a second response to the re-presented prompt. In some embodiments, intervening comprises terminating the survey without presenting prompts other than the presented prompts. In some embodiments, intervening further comprises scheduling a later time at which to re-administer the survey to the survey taker.

In another aspect, the present disclose provides a method for determining the reliability of survey responses. The method can comprise (a) obtaining, at a first time, (i) a first plurality of responses to a plurality of queries in a survey and (ii) first metadata about said first plurality of responses, which first metadata comprises at least first response times for each of said first plurality of responses; (b) obtaining, at a second time, (i) a second plurality of responses to said plurality of queries and (ii) second metadata about said second plurality of responses, which second metadata comprises at least second response times for each of said second plurality of responses; and (c) determining, based at least in part on a difference between (i) said first plurality of responses and second plurality of responses or (ii) said first metadata and said second metadata, a reliability of said first plurality of responses.

In some embodiments, (c) comprises determining whether said difference between said first metadata and said second meta data exceeds a threshold.

In some embodiments, determining whether said difference between said first metadata and said second meta data exceeds a threshold comprises determining whether an aggregate difference between said first response times and said second response times exceeds a threshold. In some embodiments, determining whether said difference between said first metadata and said second meta data exceeds a threshold comprises determining a quantity of queries for which a difference between said first response time and said second response time exceeds a threshold and determining whether said quantity exceeds a threshold.

In some embodiments, (c) comprises determining whether a difference between (i ) said first plurality of responses and second plurality of responses exceeds a threshold.

In some embodiments, determining whether a difference between (i) said first plurality of responses and second plurality of responses exceeds a threshold comprises determining a quantity of queries for which said first response differs from said second response and determining if said quantity exceeds a threshold.

In some embodiments, (c) comprises, for a query in said plurality of queries, determining whether a difference between said first response and said second response exceeds a threshold.

In some embodiments, said reliability is reduced in (c) if, for a query in said plurality of queries, said second response time is longer than said first response time.

In some embodiments, said reliability is increased in (c) if, for a query in said plurality of queries, said first response time is equal to or longer than said second response time.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a behavioral health survey analysis system in which a behavioral health survey confidence annotation machine calculates confidence in the reliability of behavioral health survey data in accordance with the present invention;

FIG. 2 is a block diagram of the behavioral health survey confidence annotation machine of FIG. 1 in greater detail;

FIG. 3 is a block diagram of survey annotation logic of the behavioral health survey confidence annotation machine of FIG. 2 in greater detail;

FIG. 4 is a block diagram of confidence annotation logic of the survey annotation logic of FIG. 3 in greater detail;

FIG. 5 is a block diagram of survey annotation system data of the behavioral health survey confidence annotation machine of FIG. 2 in greater detail;

FIG. 6 shows historical behavioral health survey data;

FIG. 7 is a logic flow diagram illustrating a two-pass administration of a behavioral health survey in accordance with the present invention;

FIG. 8 is a logic flow diagram illustrating the measurement of confidence in the reliability of survey data in accordance with the present invention;

FIGS. 9, 10, and 11 are each a logic flow diagram of a respective operation of FIG. 8 in greater detail;

FIG. 12 is a block diagram of survey data culling logic of the behavioral health survey confidence annotation machine of FIG. 2 in greater detail;

FIG. 13 is a logic flow diagram illustrating the culling of a corpus of survey taker and survey data by the survey data culling logic of FIG. 12 in accordance with the present invention;

FIG. 14 is a logic flow diagram showing an operation of the logic flow diagram of FIG. 13 in greater detail;

FIG. 15 is a logic flow diagram illustrating the identification of highly consistent and highly inconsistent survey takers by the survey data culling logic of FIG. 12 in accordance with the present invention;

FIG. 16 shows a behavioral health survey annotation system in which a behavioral health survey confidence annotation machine, a clinical data server computer system, and a survey taker device cooperate to calculate confidence in the reliability of behavioral health survey data in accordance with the present invention;

FIG. 17 is a logic flow diagram illustrating on-line administration of a behavioral health survey, the real-time annotation of confidence in the reliability of thereof, and associated intervention in the on-line behavioral health survey the survey annotation logic of FIG. 3 in accordance with the present invention; and

FIG. 18 is a block diagram of the behavioral health survey confidence annotation machine of FIG. 1 in greater detail.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2,or 1 is equivalent to less than or equal to 3, less than or equal to 2, or less than or equal to 1.

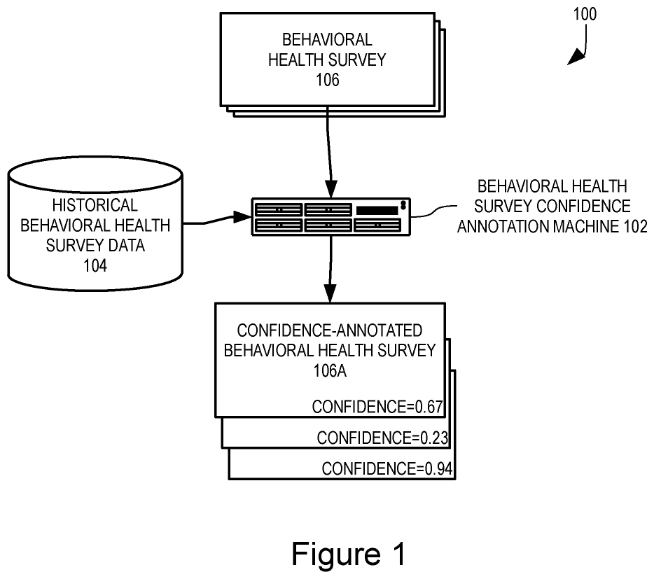

FIG. 1 schematically illustrates a behavioral health survey confidence annotation machine 102 of a behavioral health survey confidence system 100. The behavioral health survey confidence annotation machine 102 can determine a degree of confidence in the reliability of a survey taker's responses given in a behavioral health survey. The behavioral health survey confidence annotation machine 102 can receives behavioral health survey results 106 and determine a degree of confidence in the reliability of the results to produce confidence-annotated behavioral health survey results 106A. Given such a measure of confidence, any analysis of confidence-annotated behavioral health survey results 106A, e.g., statistical analysis and analysis through artificial intelligence (AI) such as deep machine learning, can be significantly more accurate and useful. For example, in such analysis, survey results with lower confidence can be weighted less or disregarded altogether while survey results with higher confidence can be weighted more heavily. The behavioral health survey confidence annotation machine 102 as described herein can be distributed across multiple computer systems.

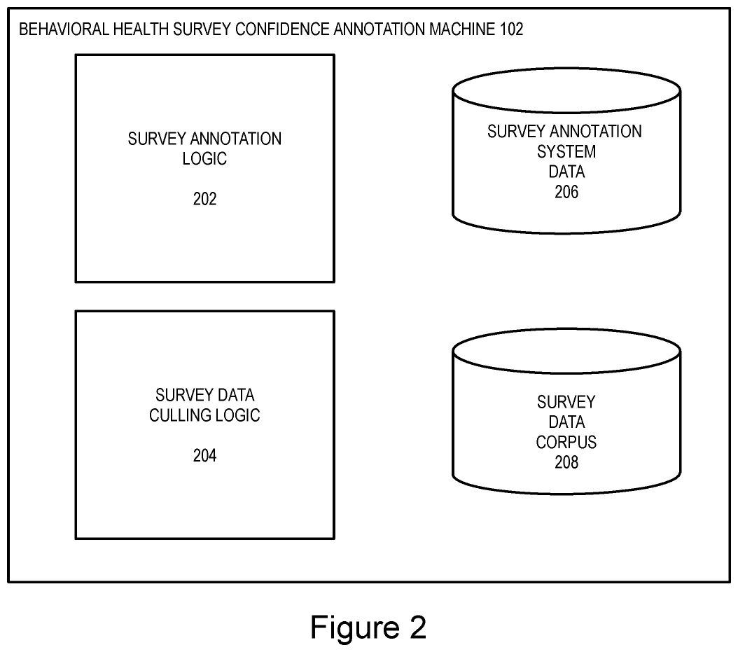

The behavioral health survey confidence annotation machine 102 is shown in greater detail in FIG. 2. The behavioral health survey confidence annotation machine 102 can include survey annotation logic 202, survey data culling logic 204, survey annotation system data 206, and survey data corpus 208.

Each of the components of the behavioral health survey confidence annotation machine 102 is described more completely below. Briefly, survey annotation logic 202 can annotate health survey results with confidence levels in the reliability of the results. Survey annotation logic 202 can also administer an interactive health survey to a human survey taker and annotates confidence levels in the reliability of the responses by the survey taker in real-time and can intervene in the administration of the behavioral health survey to improve quality of survey results. As used herein, the reliability of health survey results may be the degree to which the results accurately reflect the behavioral health state of the survey taker. Survey data culling logic 204 can identify unreliable behavioral health survey results stored in survey annotation system data 206 and remove those unreliable behavioral health survey results from consideration when analyzing such test results statistically and/or through AI. This can significantly improve such analysis. Survey analysis system data store 208 stores and maintains all survey data needed for, and collected by, analysis in the manner described herein.

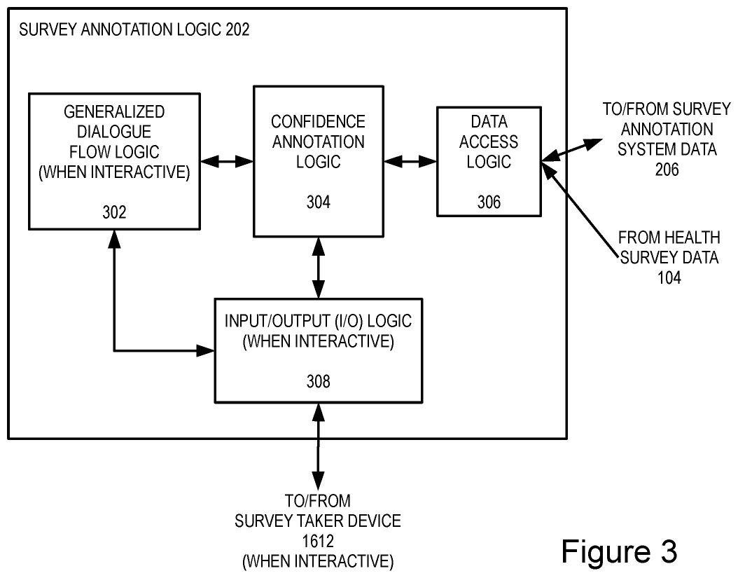

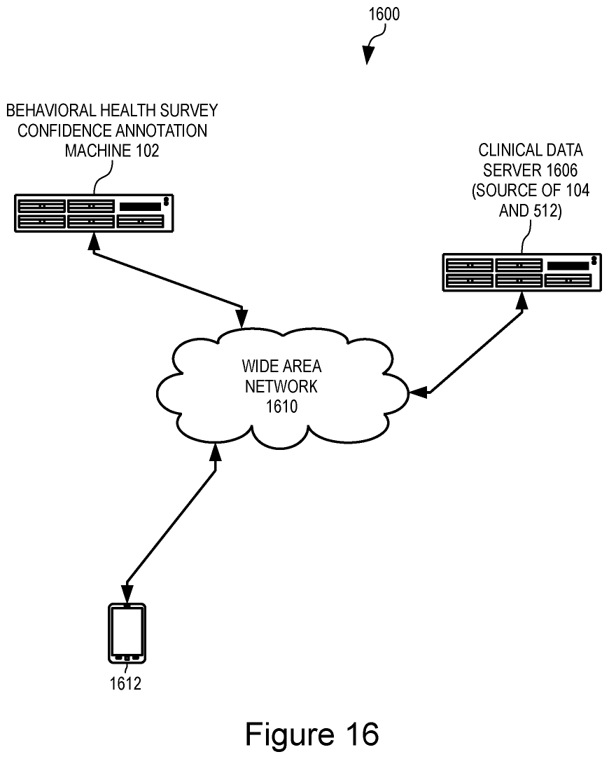

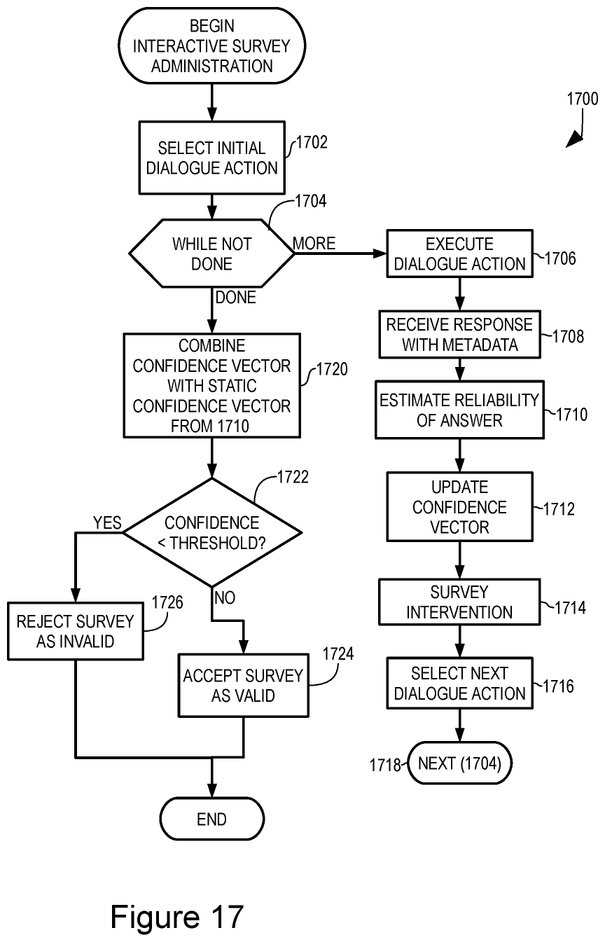

Survey annotation logic 202 is shown in greater detail in FIG. 3. Survey annotation logic 202 can include generalized dialogue flow logic 302, confidence annotation logic 304, data access logic 306, and input/output (I/O) logic 308. Generalized dialogue flow logic 302 and input/output (I/O) logic 308 can be used in embodiments in which survey annotation logic 202 administers an interactive health survey with a human survey taker in a manner described below in conjunction with FIGS. 16 and 17 and are described more completely below in conjunction therewith.

Data access logic 306 can retrieve data from, and send data to, survey annotation system data 206 to facilitate the operation of survey annotation logic 202.

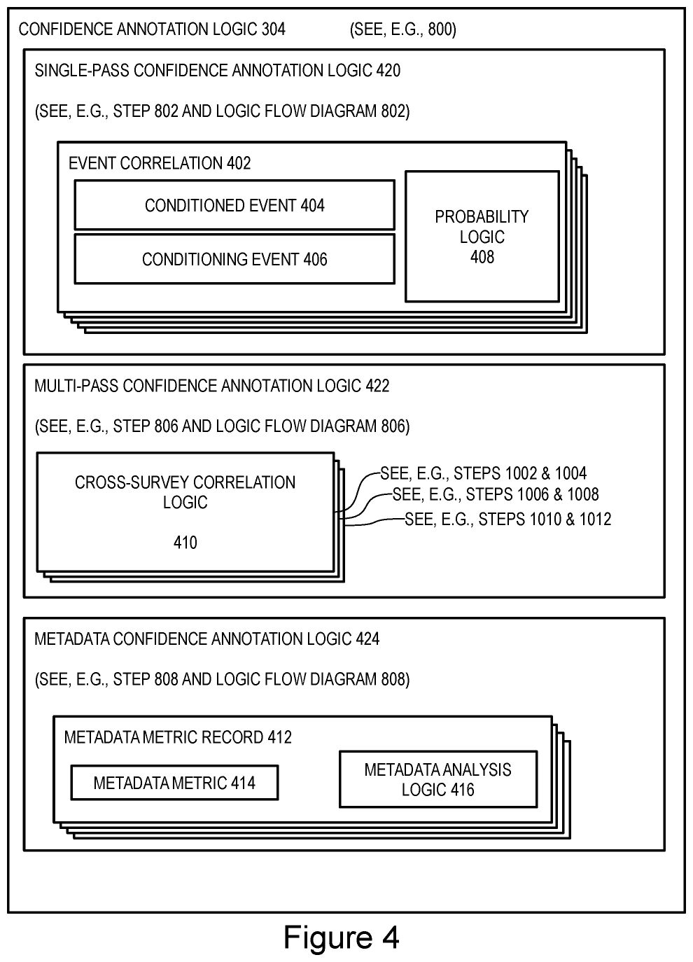

Confidence annotation logic 304 cam receive survey and survey taker data from survey annotation system data 206 and historical behavioral health survey data 104 through data access logic 306, annotate confidence levels, and store results of such analysis in survey annotation system data 206 through data access logic 306. Confidence annotation logic 304 is shown in greater detail in FIG. 4.

Confidence annotation logic 304 can include single-pass confidence annotation logic 420, multi-pass confidence annotation logic 422, and metadata confidence annotation logic 424.

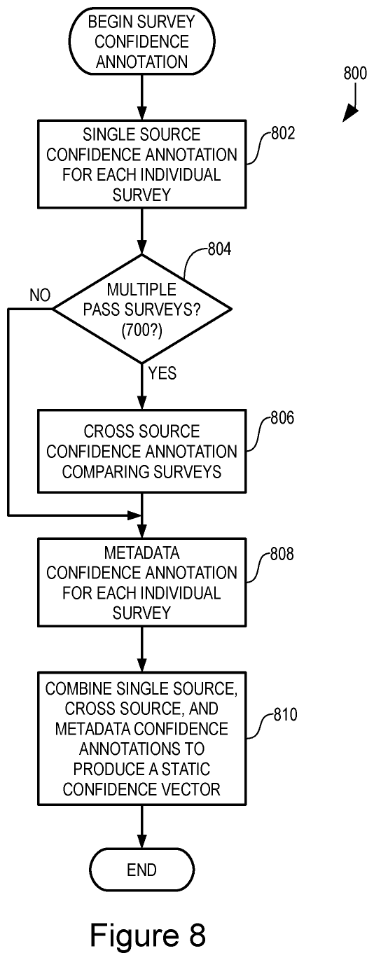

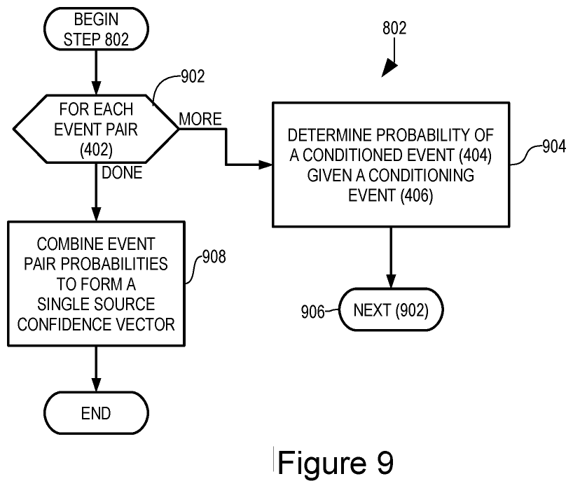

Single-pass confidence annotation logic 420 can perform single-pass confidence annotation in a manner described below in conjunction with operation 802 (FIG. 8) and logic flow diagram 802 (FIG. 9). Single-pass confidence annotation logic 420 includes a number of event correlations 402, each of which represents a pair of events and facilitates determining of the likelihood of occurrence of a conditioned event 404 given occurrence of a conditioning event 406 for confidence annotation according to probability logic 408.

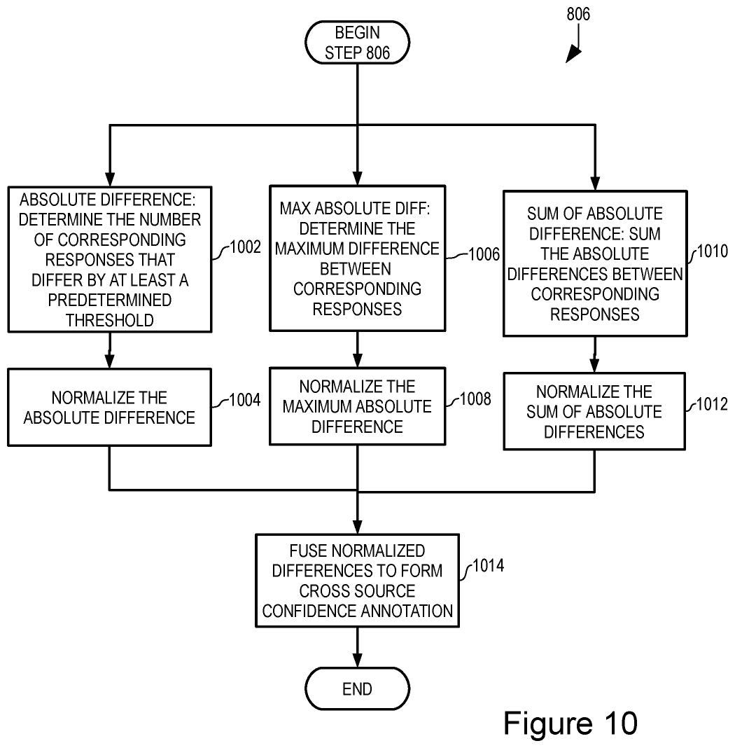

Multi-pass confidence annotation logic 422 can perform multi-pass confidence annotation in a manner described below in conjunction with operation 806 (FIG. 8) and logic flow diagram 806 (FIG. 10). Multi-pass confidence annotation logic 422 can include multiple approaches for application of cross-survey correlation logic 410 (FIG. 4), each of which can represent logic to compare multiple passes of the health survey for confidence annotation.

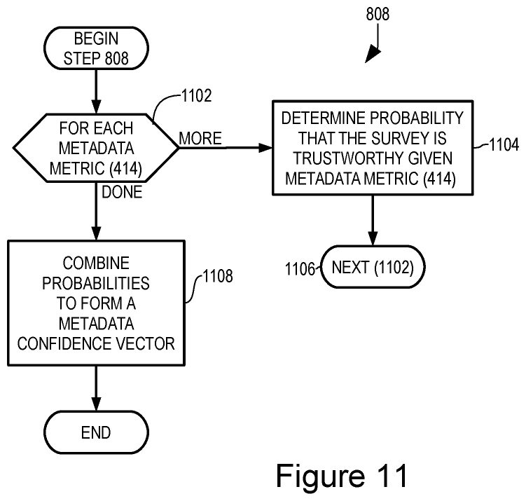

Metadata confidence annotation logic 424 can perform metadata confidence annotation in a manner described below in conjunction with operation 808 (FIG. 8) and logic flow diagram 802 (FIG. 11). Metadata confidence annotation logic 424 can include metadata metric records 412 (FIG. 4), each of which can represent a metadata metric 414 for confidence annotation according to metadata analysis logic 416. Metadata metric 414 can use any portion of survey taker metadata 532 and survey metadata 530, both of which are described below.

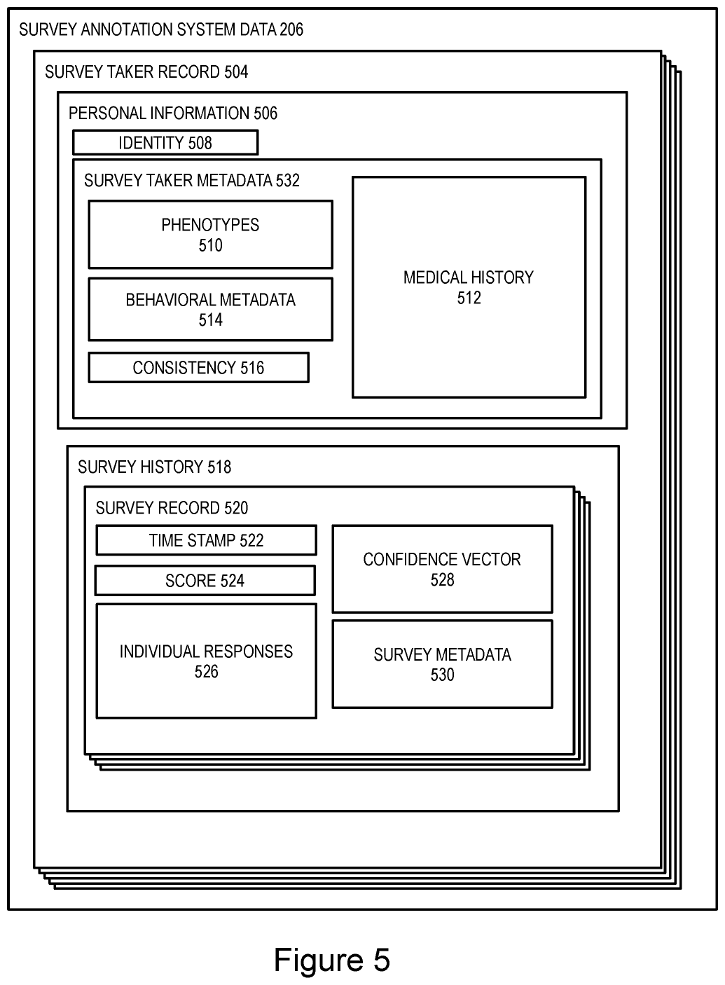

Survey annotation system data 206 (FIG. 2) is shown in greater detail in FIG. 5 and may include a number of survey taker records 504, each of which may include data representing a particular survey taker for which survey annotation logic 202 (FIG. 3) scores confidence in health survey results.

Personal information 506 (FIG. 5) of survey taker record 504 may include data that represents the subject survey taker generally and may not be specific to any behavioral health surveys. Personal information 506 may include identity 508, which may include data identifying the subject survey taker, and survey taker metadata 532. Personally identifying information is not needed in identity 508 so long as each survey taker can be identified uniquely among all survey takers represented in survey annotation system data 206. Survey taker metadata 532 can store generally any type of information about the survey taker other than identifying data.

For example, phenotypes 510 may include data representing various phenotypes of the subject survey taker. Such phenotypes can include, for example, gender, age (or date of birth), nationality, marital status, income, ethnicity, and language(s) (including a degree of proficiency in each). Medical history 512 may include data representing a medical history of the subject survey taker (e.g., medical conditions, past surgeries, lab test results, family history, genetic information, etc.). Behavioral metadata 514 may include data representing behavior of the user and can include such things as typing speed, reading speed, etc. Consistency 516 may include data representing whether the subject survey taker consistently provides reliable results of health surveys.

Survey history 518 may include data representing prior health surveys, including a number of survey records 520, each of which may represent a prior health survey taken by the subject survey taker. Results of a health survey analysis by survey annotation logic 202 may be recorded in a survey record 520 as described below.

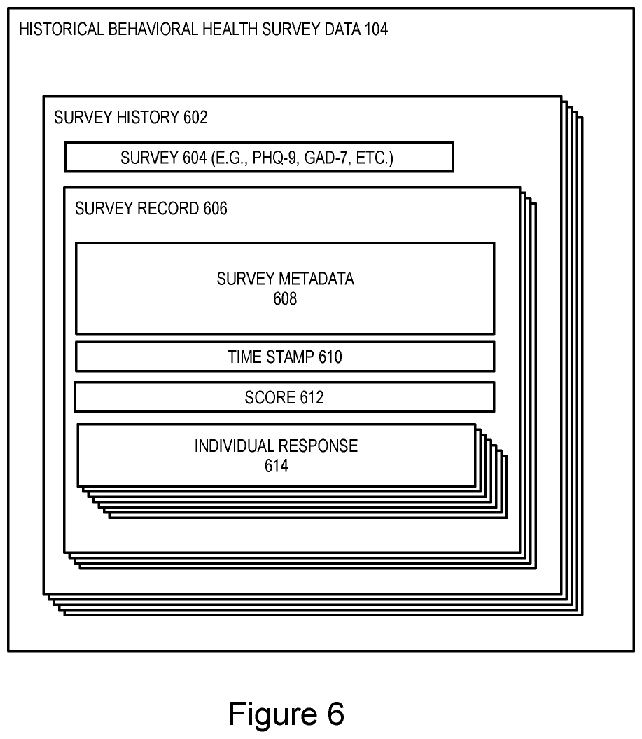

Historical behavioral health survey data 104 is shown in greater detail in FIG. 6. Historical behavioral health survey data 104 may represent behavioral survey data available from third-party sources. Accordingly, the particular format and content of historical behavioral health survey data 104 can vary widely from source to source. FIG. 6 represents the general overall nature of available behavioral health survey data to facilitate understanding and appreciation of the present invention.

Historical behavioral health survey data 104 may include a number of survey histories 602, each of which may correspond to a particular type of behavioral health survey, which is identified by survey 604.

Each of survey histories 602 may include a number of survey records 606, each of which may represent a completed survey of the type identified by survey 604. Survey metadata 608 may include data representing one or more attributes of a survey that are not represented in other fields of survey record 606. Survey metadata 608 can include information about the particular human taker of the survey, such as the age, gender, and ethnicity of the taker, for example. Survey metadata 608 can include other metadata of the survey such as whether and how much compensation was provided to the survey taker and the environment or platform in which the survey was given, for example.

Time stamp 608 may represent the date and time of completion of the survey. Score 612 may represent the overall score of the subject completed survey. Individual responses 614 may each represent an individual survey response by the survey taker in the subject completed survey.

It should be appreciated that, since the particular format and content of historical behavioral health survey data 104 can vary widely from source to source, various portions of survey record 606 may be missing, though ordinarily at least score 612 is included. Availability and content of survey metadata 608 particularly widely across sources of historical behavioral health survey data 104. It should also be noted that, while surveys represented by survey records 606 are referred to as completed surveys, “completed surveys” as used herein are surveys for which the survey taker has ceased taking the survey, even if the survey taker has not responded to all prompts of the survey. Thus, even if the survey taker did not respond to all prompts of the survey, administration of the survey is considered completed.

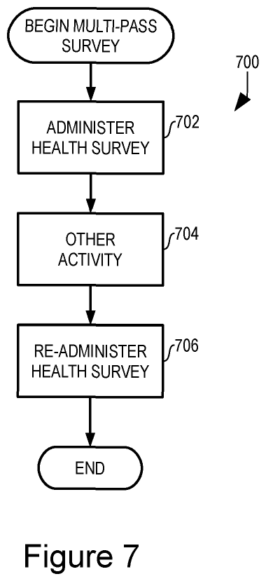

As described above, confidence annotation logic 304 (FIG. 4) may include multi-pass confidence annotation logic 422 for analyzing results of multi-pass health surveys. Multiple-pass health surveys may provide especially good insight into the reliability of results of a health survey. Such a multiple-pass health survey is illustrated by logic flow diagram 700 (FIG. 7).

In operation 702, the behavioral health survey may be administered to the survey taker. The behavioral health survey can be administered by survey annotation logic 202 in the manner described below in conjunction with logic flow diagram 1700 (FIG. 17). In operation 704, the survey taker may be engaged in activity that is not part of the survey of operation 702. This other activity may serve to distract the survey taker from perfectly remembering his or her answers to the first instance of the survey. It can be used for any practical additional purpose such as to gather useful information unrelated to the survey itself. In operation 706, the behavioral health survey may be administered to the survey taker again (i.e., a second time). Operations 704 and 706 can be performed by survey annotation logic 202. Comparison of the two passes can help measure the confidence in the reliability of the survey taker's responses in the manner described below.

Logic flow diagram 800 (FIG. 8) illustrates the measurement of confidence in the reliability of the survey taker's responses in a behavioral health survey after completion of the behavioral health survey by survey annotation logic 202. In operation 802, confidence annotation logic 304 can evaluate confidence in the reliability of the results of the behavioral health survey for each individual pass of the behavioral health survey. Operation 802 is shown in greater detail as logic flow diagram 802 (FIG. 9) and is described below.

In test operation 804 (FIG. 8), confidence annotation logic 304 can determine whether a multiple-pass behavioral health survey was administered in the manner described above with respect to logic flow diagram 700 (FIG. 7). Multi-pass behavioral health surveys are not always administered. If a multiple-pass behavioral health survey was administered, confidence annotation logic 304 can perform cross-source confidence evaluation by comparing the multiple surveys in operation 806 (FIG. 8) as described below in greater detail in conjunction with logic flow diagram 806 (FIG. 10). Conversely, if confidence annotation logic 304 determines that only a single-pass behavioral health survey was administered, confidence annotation logic 304 can skips operation 806 (FIG. 8).

In operation 808, confidence annotation logic 304 can use metadata to evaluate confidence in the reliability of the results of the health survey. Operation 802 is shown in greater detail as logic flow diagram 802 (FIG. 9) and is described below.

In operation 810 (FIG. 8), confidence annotation logic 304 can combine the confidence evaluations from operations 802, 806, and 808 to produce a static confidence vector, e.g., confidence vector 528 (FIG. 5). As described below, this static confidence vector can be combined with the intermediate confidence vector in operation 1720 (FIG. 17).

Operation 802 (FIG. 8) is shown in greater detail as logic flow diagram 802 (FIG. 9). Loop operation 902 and next operation 906 define a loop in which confidence annotation logic 304 can process each of a number of observed event pairs of the health survey in operation 904. Each of the observed event pairs may correspond to a pair of events observed in the survey taker's responses corresponding to conditioned event 404 (FIG. 4) and a conditioning event 406 of any of event correlations 402. During each iteration of the loop of operations 902-906 (FIG. 9), the particular one of event correlations 402 processed by confidence annotation logic 304 is sometimes referred to as the subject event correlation.

In operation 904, confidence annotation logic 304 can determine the probability that conditioned event 404 (FIG. 4) is observed given observation of conditioning event 406 using probability logic 408. For example, suppose conditioned event 404 represents a PHQ-9 score of less than five (5) and the conditioning event 406 represents a response of three (3) on the second question of the PHQ-9. The PHQ-9 has nine (9) questions and responses range from zero (0) to three (3), so a score of four (4) or less with a response of three (3) on any question means that at most one other question had a response of one (1) and all other questions had responses of zero (0), which generally has a low probability.

Probability logic 408 can determine the probability that conditioned event 404 is observed when conditioning event 406 is also observed. In this illustrative embodiment, probability logic 408 can be configured using statistical analysis of an entire corpus of survey data. For example, confidence annotation logic 304 can find all PHQ-9 health surveys with a score of no more than four as represented in score 524 (FIG. 5) of survey history 518 of all survey taker records 504 and/or in score 612 (FIG. 6) of all survey records 606 of historical behavioral health survey data 104 and, from those, determine how many of those have a response of three for the second question as represented in individual responses 526 and/or in individual responses 614. Confidence annotation logic 304 (FIG. 4) can configure probability logic 408 to respond with the ratio of the latter to the former.

Another example of an event pair is a particular score in the PHQ-9 as conditioned event 404 and a particular score in the GAD-7 as conditioning event 406. In addition to survey history 518 (FIG. 5) of all survey taker records, the data corpus can include health survey scores and responses represented in medical history 512 as well as survey records 606 to the extent that survey taker metadata 608 (FIG. 6) can identify survey records 606, across all survey histories 602, representing completed surveys taken by the same survey taker. In addition, the corpus can be culled in a manner described below to remove unreliable behavioral health survey results from consideration. The corpus is unlikely to change often, so confidence annotation logic 304 (FIG. 4) can update probability logic 408 relatively infrequently, e.g., weekly, monthly, whenever the size of the corpus increases by an appreciable amount (e.g., 2%), or after each culling of the corpus as described below.

Event correlations 402 can be included in logic within survey taker device 1612 (FIG. 16) such that interactive administration of the health survey with real-time confidence checking can be performed by survey taker device 1612 when off-line, i.e., not in communication with behavioral health survey confidence annotation machine 102 through WAN 1610. The same is true for cross-survey correlation logic 410 (FIG. 4) and metadata metric records 412, both of which are described below.

After operation 904 (FIG. 9), processing may transfer through next operation 906 to loop operation 902 in which the next observed event pair is processed by confidence annotation logic 304 according to the loop of operations 902-906. When all of the observed event pairs have been processed by confidence annotation logic 304, processing transfers from loop operation 902 to operation 908.

In operation 908, confidence annotation logic 304 can combine all probabilities determined in operation 904 to form a single-source (e.g., from a single pass of a behavioral health survey) confidence vector. In this illustrative embodiment, confidence annotation logic 304 can include each probability determined in each performance of operation 904 as one dimension in the single-source confidence vector.

Operation 806 is shown in greater detail as logic flow diagram 806 (FIG. 10). Operations 1002, 1006, and 1010 each correspond to a respective instance of cross-survey correlation logic 410 (FIG. 4). Each cross-survey correlation logic 410 can process at least two (2) passes of the same health survey given contemporaneously in the manner described above with respect to logic flow diagram 700 (FIG. 7).

In operation 1002 (FIG. 10), confidence annotation logic 304 can determine the number of responses to corresponding prompts that changed by at least a predetermined threshold between the multiple passes using a first instance of cross-survey correlation logic 410 (FIG. 4). For example, confidence annotation logic 304 can determine the number of responses to corresponding prompts that changed by two (2) or more between the multiple passes.

In operation 1004 (FIG. 10), confidence annotation logic 304 can normalize the number determined in operation 1002 to be a real number in the range of 0.0 to 1.0. Normalization in operation 1004 can be accomplished in any of a number of ways. In one illustrative embodiment, confidence annotation logic 304 can analyze the same corpus described above with respect to operation 904 (FIG. 9) to determine a percentile for each of the possible results from operation 1002 (FIG. 10). If the health survey is the PHQ-9, there are nine (9) possible results. If the health survey is the GAD-7, there are seven (7) possible results. The respective percentiles and the corresponding results from operation 1002 can be represented in the first instance of cross-survey correlation logic 410 (FIG. 4) as a simple lookup table.

In operation 1006 (FIG. 10), confidence annotation logic 304 can determine the greatest absolute difference between responses to corresponding prompts of the multiple passes using a second instance of cross-survey correlation logic 410 (FIG. 4).

In operation 1008 (FIG. 10), confidence annotation logic 304 can normalize the number determined in operation 1006 to be a real number in the range of 0.0to 1.0. Normalization in operation 1008 can be accomplished in any of a number of ways. In one illustrative embodiment, confidence annotation logic 304 can analyze the same corpus described above with respect to operation 904 (FIG. 9) to determine a percentile for each of the possible results from operation 1006 (FIG. 10). If the health survey is the PHQ-9, responses range from zero (0) to three (3), so there are four (4) possible results. The respective percentiles and the corresponding results from operation 1006 can be represented in the second instance of cross-survey correlation logic 410 (FIG. 4) as a simple lookup table.

In operation 1010 (FIG. 10), confidence annotation logic 304 can determine the sum of absolute differences between corresponding prompts of the multiple passes using a third instance of cross-survey correlation logic 410 (FIG. 4).

In operation 1012 (FIG. 10), confidence annotation logic 304 can normalize the number determined in operation 1010 to be a real number in the range of 0.0 to 1.0. Normalization in operation 1012 can be accomplished in any of a number of ways. In one illustrative embodiment, confidence annotation logic 304 can analyze the same corpus described above with respect to operation 904 (FIG. 9) to determine a percentile for each of the possible results from operation 1010 (FIG. 10). Similar to normalization in operations 1004 and 1008, there are a relatively few possible results from operation 1010. Accordingly, the respective percentiles and the corresponding results from operation 1010 can be represented in the third instance of cross-survey correlation logic 410 (FIG. 4).

In operation 1014, confidence annotation logic 304 can fuse the normalized values resulting from operations 1004, 1008, and 1012 to form a cross-source confidence vector. Confidence annotation logic 304 can include each of the normalized values resulting from operations 1004, 1008, and 1012 as one dimension in the cross-source confidence vector. In an alternative embodiment, confidence annotation logic 304 can fuse the normalized values resulting from operations 1004, 1008, and 1012 to form a cross-source confidence scalar value by computing a value representative of the normalized values as a whole. Examples of such computing can include, for example, weighted linear and nonlinear combination including statistical voting, local regression, simple regression, and so on.

Operation 808 is shown in greater detail as logic flow diagram 808 (FIG. 11). Loop operation 1102 and next operation 1106 define a loop in which confidence annotation logic 304 can process each of metadata metric records 412 (FIG. 4) in operation 1104. During each iteration of the loop of operations 1102-1106 (FIG. 11), the particular one of metadata metric records 412 (FIG. 4) processed by confidence annotation logic 304 is sometimes referred to as the subject metadata metric record.

In operation 1104 (FIG. 11), confidence annotation logic 304 can determine the probability that the subject health survey results are reliable according to metadata metric 414 (FIG. 4) of the subject metadata metric record using probability logic 416 of the subject metadata metric record. For example, suppose metadata metric 414 represents the duration of the survey taker's delay before responding to the first prompt of the health survey. It has been observed that the longer initial delay, the more reliable the results of the health survey, perhaps representing greater consideration of the behavioral health survey before beginning to respond. Accordingly, metadata analysis logic 416 of the same metadata metric record 412 scores greater confidence that the results of the behavioral health survey are reliable when the initial delay is longer.

Metadata analysis logic 416 can determine the probability that the subject behavioral health survey results are reliable according to metadata metric 414 of the subject metadata metric record. In this illustrative embodiment, metadata analysis logic 416 may be configured using statistical analysis of the same corpus described above with respect to operation 904 (FIG. 9). For example, confidence annotation logic 304 (FIG. 4) can find all health surveys of survey history 518 (FIG. 5) of all survey taker records 504 and correlate confidence vectors 528 with the length of the initial delay as presented in survey metadata 530. Confidence annotation logic 304 (FIG. 4) can also use the same data from survey metadata 608 (FIG. 6) to the extent such data is available.

There are numerous other illustrative examples of metadata metrics that can be represented by metadata metric 414 including the following. Delays prior to responding to other prompts of the health survey as well as the overall duration of the health survey can be metadata metrics. It has been observed that longer and more varied delays in responding to the various prompts, as well as longer test durations, indicate more reliable results of the health survey, suggesting more deliberately considered responses. The number of corrections made by the survey taker to previously given responses may also indicate a more deliberate consideration of the behavioral health survey. Deviations in the order of responses given by the survey taker from the order in which the prompts are presented to the survey taker may similarly indicate greater attention and careful consideration. In embodiments in which survey taker device 1612 (FIG. 16) captures audio and/or video signals of the survey taker during administration of the behavioral health survey, such signals can be analyzed for gaze and eye tracking as well as analysis of vocal responses to the prompts. Metadata analysis logic 416 (FIG. 4) can also analyze metadata metric 414 in the context of the survey taker's behavior when not taking the health survey, e.g., using extra-survey metadata 514 (FIG. 5) and, to the extent such data is available, survey metadata 608 (FIG. 6).

In addition to user interface metadata, confidence annotation logic 304 (FIG. 4) can use survey taker metadata, e.g., as represented in phenotypes 510 (FIG. 5) and/or medical history 512, as metrics to analyze confidence in the reliability of responses of a given behavioral health survey. Just a few illustrative examples of the possible survey taker metadata metrics include age, gender, ethnicity, location, time of day, collection platform, electronic medical record (EMR) data, claims data, survey taker history, and past survey scores.

After operation 1104 (FIG. 11), processing transfers through next operation 1106 to loop operation 1102 in which the next observed event pair is processed by confidence annotation logic 304 according to the loop of operations 1102-1106. When all of the metadata metric records have been processed by confidence annotation logic 304, processing transfers from loop operation 1102 to operation 1108.

In operation 1108, confidence annotation logic 304 can combine all probabilities determined in operation 1104 to form a metadata confidence vector. In this illustrative embodiment, confidence annotation logic 304 can include each probability determined in each performance of operation 1104 as one dimension in the metadata confidence vector.

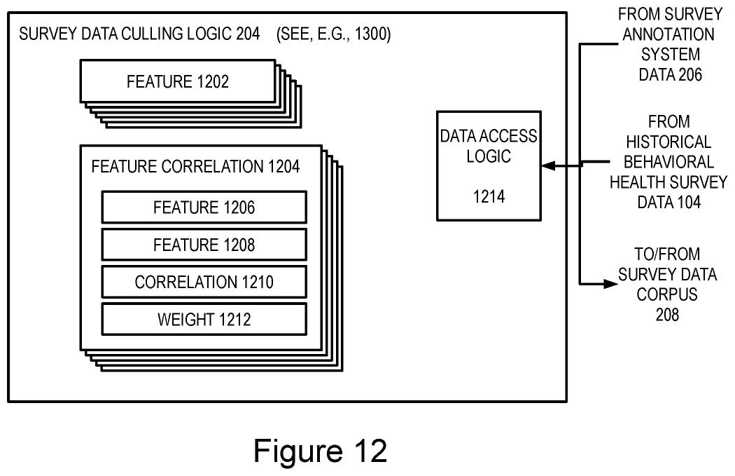

As described above, survey data culling logic 204 can identify low-confidence behavioral health survey results stored in survey data corpus 208 and remove those low-confidence health survey results from consideration when analyzing such survey results statistically and/or through AI. Survey data corpus 208 may represent that portion of historical behavioral health survey data 104 (FIG. 6) and survey annotation system data 206 (FIG. 5) used by survey annotation logic 202 in the manner described above. Survey data corpus 208 can include copies of portions of historical behavioral health survey data 104 and survey annotation system data 206 and/or can include such data by reference thereto. Survey data culling logic 204 is shown in greater detail in FIG. 12.

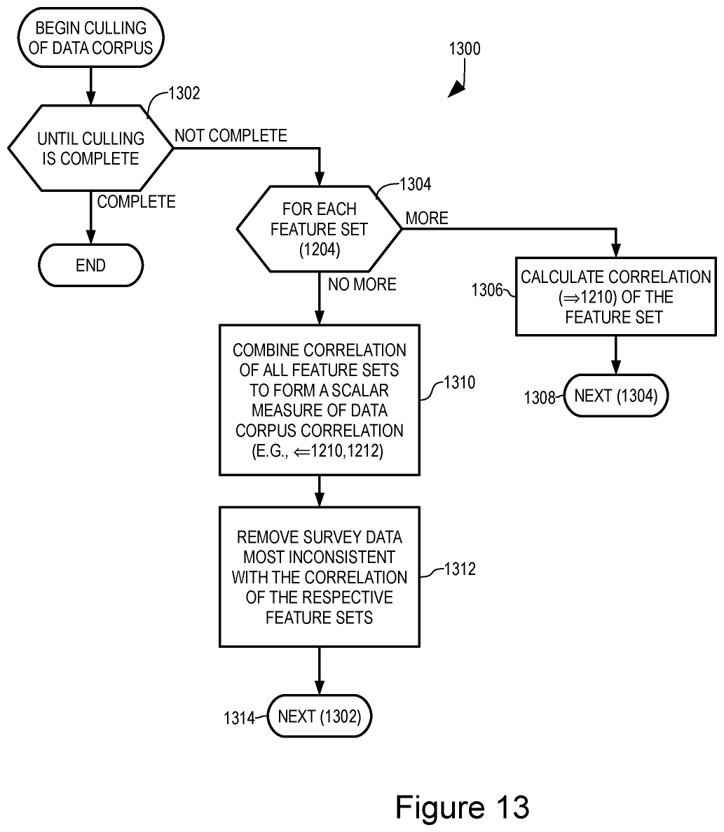

Survey data culling logic 204 may include a number of features 1202, a number of feature correlations 1204, and data access logic 1214. Data access logic 1214 can retrieve data from, and send data to, survey annotation system data 206 to facilitate operation of survey data culling logic 204. Each of features 1202 may represent any item of information in survey taker records 504. Features 1202 may be selected in a manner described more completely below. Feature correlations 1204 may represent sets of two or more of features 1202 and are described more completely below in the context of logic flow diagram 1300 (FIG. 13). In this illustrative embodiment, feature correlations 1204 represent pairs of features 1202.

The manner in which survey data culling logic 204 (FIG. 2) culls survey data corpus 208 is illustrated in logic flow diagram 1300 (FIG. 13). Loop operation 1302 and next operation 1314 may define a loop in which survey data culling logic 204 iteratively culls survey data corpus 208 according to operations 1304-1312 until the culling is deemed complete. In one embodiment, survey data culling logic 204 may deem culling to be complete when the overall measure of confidence in the reliability of all health survey results of survey data corpus 208 is at least a predetermined minimum threshold. In an alternative embodiment, survey data culling logic 204 may deem culling to be incomplete as long as the size of survey data corpus 208 is above a predetermined statistically acceptable size. In yet another embodiment, survey data culling logic 204 may deem culling to be incomplete when further iterations of the loop of operations 1302-1314 fail to significantly improve the overall measure of confidence in the reliability of all health survey results of survey data corpus 208. Until culling is complete, processing transfers from loop operation 1302 to loop operation 1304.

Loop operation 1304 and next operation 1308 may define a loop in which survey data culling logic 204 processes each of feature correlations 1204 (FIG. 12) according to operation 1306 (FIG. 13). During a given iteration of the loop of operations 1304-1308, the particular one of the feature correlations 1204 processed by survey data culling logic 204 is sometimes referred to as the subject feature correlation.

In operation 1306, survey data culling logic 204 can calculate correlation 1210 (FIG. 12) for the features of the subject feature correlation. Correlation 1210 can be any type of measurement of relationships between two or more of features 1202. Examples of such relationship measurements include correlation, mutual information, and measurements based on neural-network models such as graphical models. In this illustrative embodiment, correlation 1210 represents mutual information of two features, i.e., features 1206 and 1208. Feature 1206 identifies one of features 1202, and feature 1208 identifies a different one of features 1202. In this illustrative embodiment, a feature correlation 1204 exists for each and every unique combination of features 1202. Survey data culling logic 204 stores the calculated correlation in correlation 1210 of the subject feature correlation.

After calculating correlation 1210 for the features of the set of the subject feature correlation in operation 1306 (FIG. 13), processing by survey data culling logic 204 can transfer through next operation 1308 to loop operation 1304 and survey data culling logic 204 can process the next of feature correlations 1204 according to the loop of operations 1304-1308. When all of feature correlations 1204 have been processed, processing transfers from loop operation 1304 to operation 1310.

In operation 1310 (FIG. 13), survey data culling logic 204 can combine all correlations 1210 (FIG. 12) to form a scalar measure of data quality of survey data corpus 208. In this illustrative embodiment, survey data culling logic 204 can combine all correlations 1210 by calculating a weighted mean in which each correlation 1210 is weighted by a corresponding weight 1212. Weights 1212 may be configured in a manner described more completely below.

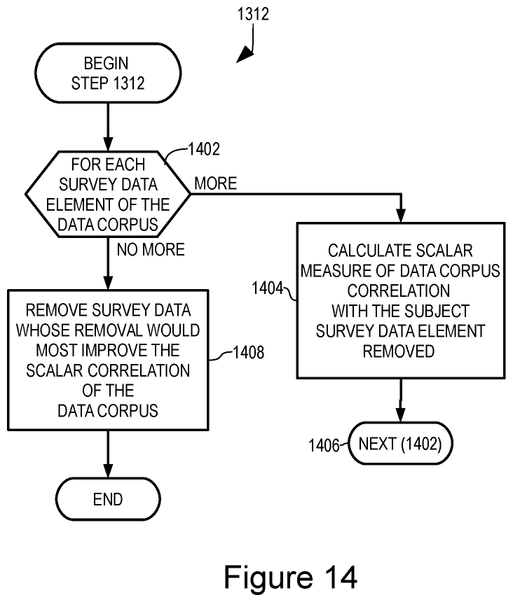

In operation 1312 (FIG. 13), survey data culling logic 204 can remove from survey data corpus 208 survey data that is most inconsistent with the correlation calculated in operation 1306. Operation 1312 is shown in greater detail as logic flow diagram 1312 (FIG. 14).

Loop operation 1402 and next operation 1406 define a loop in which survey data culling logic 204 can process each survey data element according to operation 1404. In this illustrative embodiment, survey data elements are each a survey record 520 (FIG. 5). During a given iteration of the loop of operations 1402-1406, the particular survey record 520 processed by survey data culling logic 204 is sometimes referred to as the subject survey record.

In operation 1404 (FIG. 14), survey data culling logic 204 can calculate the scalar measure of data corpus correlation in the manner described above with respect to operation 1310 (FIG. 13) but excludes the subject survey record from the calculation. In effect, survey data culling logic 204 can remove the subject survey record from the scalar measure of correlation of the data corpus to calculate what the scalar measure of data corpus correlation would be if the subject survey record were removed from survey data corpus 208.

After operation 1404 (FIG. 14), processing by survey data culling logic 204 may transfer through next operation 1406 to loop operation 1402 in which the next survey data element is processed by survey data culling logic 204 according to the loop of operations 1402-1406. When all survey data elements have been processed by survey data culling logic 204, processing transfers from loop operations 1402 to operation 1408.

In operation 1408, survey data culling logic 204 can remove the survey data elements whose removal would most improve the scalar measure of correlation of survey data corpus 208. In particular, survey data culling logic 204 can rank the survey data elements according to their respective scalar measures of data corpus correlation as calculated in operation 1404 and remove from survey data corpus 208 those survey data elements with the highest scalar measures calculated in operation 1404.

After operation 1408, logic flow diagram 1312, and therefore operation 1312 (FIG. 13), completes. From operation 1312, processing by survey data culling logic 204 transfers through next operation 1314 to loop operation 1302 and operations 1304-1312 are repeated until survey data culling logic 204 determines that culling is complete. When culling is complete, processing according to logic flow diagram 1300 completes.

Features 1202 (FIG. 12) and weights 1212 may be selected to maximize confidence in the reliability of survey taker responses to behavioral health survey prompts. Such a data corpus is sometimes referred to as a high-quality data corpus. Features 1202 and weighs 1212 can be selected automatically. Survey data culling logic 204 can be configured to perform statistical analysis of various data fields of survey taker record 504 (FIG. 5) with respect to the data corpus to identify data fields with strong correlation with survey data quality. Data fields with such strong correlations can be some of features 1202 (FIG. 12).

Weights 1212 correlate to an expected mutual information 1210. In other words, feature correlations 1204 whose correlation 1210 is expected to be relatively high in a high-quality data corpus may be attributed a greater weight 1212. As with features 1202, weights 1212 can be improved by trial and error such that the scalar measure of data quality of the data corpus more accurately represents the quality of the data corpus.

Thus, by removing low-quality survey data from survey data corpus 208, survey data culling logic 204 may significantly improve the quality of survey data corpus 208 and, as a result, any statistical or AI analysis of survey data corpus 208. For example, the higher-quality data corpus may significantly improve the measuring of confidence in the reliability of responses to health surveys in the manner described above.

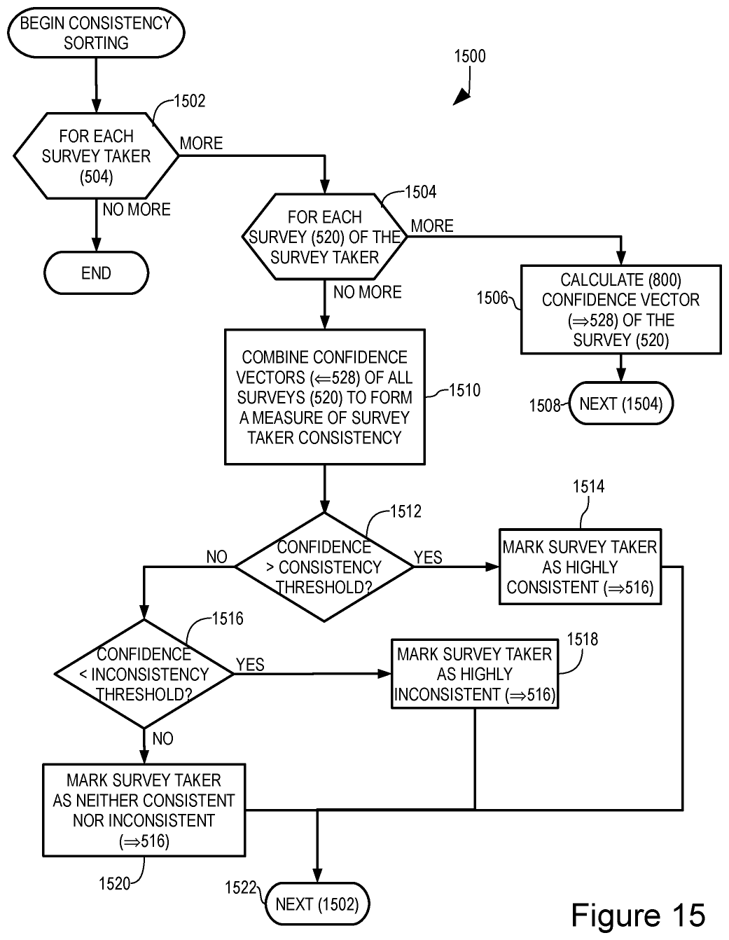

Survey data culling logic 204 (FIG. 2) may identify survey takers whose survey results are highly consistently reliable and survey takers whose survey results' reliability is inconsistent in a manner illustrated by logic flow diagram 1500 (FIG. 15).

Loop operation 1502 and next operation 1522 define a loop in which survey data culling logic 204 can process each of survey taker records 504 (FIG. 5) according to operations 1504-1520 (FIG. 15). During an iteration of the loop of operations 1502-1522, the particular one of survey taker records 504 (FIG. 5) processed by survey data culling logic 204 is sometimes referred to as the subject survey taker record, and the survey taker represented by the subject survey taker record is sometimes referred to as the subject survey taker.

Loop operation 1504 and next operation 1508 define a loop in which survey data culling logic 204 can process each of survey records 520 (FIG. 5) of the subject survey taker record according to operation 1506 (FIG. 15). During an iteration of the loop of operations 1504-1508, the particular one of survey records 520 (FIG. 5) processed by survey data culling logic 204 is sometimes referred to as the subject survey record.

In operation 1506 (FIG. 15), survey data culling logic 204 calculates a confidence vector representing a degree of confidence in the reliability of the results of the subject survey record in the manner described above with respect to logic flow diagram 800 (FIG. 8). After operation 1506, processing transfers through next operation 1508 to loop operation 1504 and survey data culling logic 204 processes the next of survey records 520 of the subject survey taker record. When all survey records 520 of the subject survey taker record have been processed according to the loop of operations 1504-1508, processing by survey data culling logic 204 transfers to operation 1510.

In operation 1510, survey data culling logic 204 can combine confidence vectors 528 calculated in the loop of operations 1504-1508 to form a single measure of consistency of the responses of the subject survey taker. Examples of such combination include, for example, weighted linear and nonlinear combination, local regression, simple regression, and mathematical voting.

In test operation 1512, survey data culling logic 204 can compare the single measure of consistency of the responses of the subject survey taker to a predetermined high consistency threshold. If the single measure of consistency is greater than the predetermined high consistency threshold, survey data culling logic 204 can mark the subject survey taker as highly consistent by storing data so indicating in consistency 516 (FIG. 5) of the subject survey taker record in operation 1514 (FIG. 15). Conversely, if the single measure of consistency is not greater than the predetermined high consistency threshold, processing transfers to test operation 1516.

In test operation 1516, survey data culling logic 204 can compare the single measure of consistency of the responses of the subject survey taker to a predetermined high inconsistency threshold. If the single measure of consistency is less than the predetermined high inconsistency threshold, survey data culling logic 204 can mark the subject survey taker as highly inconsistent by storing data so indicating in consistency 516 (FIG. 5) of the subject survey taker record in operation 1518 (FIG. 15). Conversely, if the single measure of consistency is not less than the predetermined high inconsistency threshold, survey data culling logic 204 can mark the subject survey taker as neither highly consistent nor highly inconsistent by storing data so indicating in consistency 516 (FIG. 5) of the subject survey taker record in operation 1520 (FIG. 15).

From any of operations 1514, 1518, and 1520, processing by survey data culling logic 204 transfers through next operation 1522 to loop operation 1502 and survey data culling logic 204 processes the next of survey taker records 504 (FIG. 5) according to the loop of operations 1502-1522. It should be appreciated that, while operations 1512-1520 show an illustrative embodiment in which there are three (3) categories of survey taker consistency, alternative embodiments can sort survey takers into fewer or more categories or simply store the result of operation 1510 to annotate survey taker record 504 ((5) with consistency 516, allowing custom consistency filters to be constructed for particular purposes of the users of survey annotation system data 206.

Thus, survey data culling logic 204 can identify survey takers who tend to be highly consistent in their survey responses and those who tend to be highly inconsistent. Knowing whether a given survey taker tends to be consistently reliable can be useful in both (i) annotating confidence levels in survey results from the survey taker and (ii) culling survey data in the manner described above. For example, metadata confidence annotation logic 424 (FIG. 4) can include a metadata metric record 412 that has consistency 516 (FIG. 5) as its metadata metric 414 and metadata analysis logic 416 can estimate a probability that survey results are reliable from consistency 516. This would cause consistency 516 to influence metadata confidence evaluation in operation 808 and therefore the static confidence vector produced in operation 810. In addition, since confidence vectors can be used in the manner described below to influence administration of, and processing of results of, a behavioral health survey, consistency 516 influences such administration and processing.

Moreover, identifying a significant number of survey takers whose survey results are highly inconsistent can be very helpful in improving behavioral surveys themselves or at least their administration to survey takers. In particular, statistical and/or AI analysis of survey taker records 504 for survey takers marked as highly inconsistent can identify aspects of behavioral health surveys that fail to elicit more reliable results. Correcting such aspects can significantly improve the reliability of behavioral health surveys overall.

As described above, behavioral health survey confidence annotation machine 102 can interactively administer behavioral health surveys to survey takers. A behavioral health survey confidence system 1600 (FIG. 16) in which behavioral health survey confidence annotation machine 102 interactively administers behavioral health surveys to survey takers is shown in FIG. 16. Historical behavioral health survey data 104 (FIG. 1) is available from a number of clinical data servers such as clinical data server 1606 (FIG. 16) though a wide area network (WAN) 1610, that is the Internet in this illustrative embodiment. Behavioral health survey confidence annotation machine 102 may act as a server computer system that administers the health survey with the survey taker through survey taker device 1612 through WAN 1610 and determines confidence level annotation in the manner described above.

As described briefly above, survey annotation logic 202 (FIG. 3) may include generalized dialogue flow logic 302 and I/O logic 308. I/O logic 308 may affect administration of the health survey by sending prompt data to, and receiving responsive data from, survey taker device 1612. Some or all of the prompt data causes survey taker device 1612 to present prompts to the survey taker. For example, if the health survey is a form of the PHQ-9 health survey, the prompt data can cause survey taker device 1612 to present the various questions of the PHQ-9 test to the survey taker. The responsive data may represent responses by the survey taker to the presented prompts. The responsive data can also include metadata such as user interface data as described more completely above. I/O logic 308 can receive data from generalized dialogue flow logic 302 that specifies prompts to be presented to the survey taker and sends prompt data representing those prompts to survey taker device 1612.

Generalized dialogue flow logic 302 can conduct the health survey with the human survey taker by determining what prompts I/O logic 308 should present to the survey taker and receive the response data from I/O logic 308 to determine (i) which prompt should be presented next, if any, and (ii) when the behavioral health survey is complete.

In this illustrative embodiment, survey annotation logic 202 can administer a health survey in the manner illustrated in logic flow diagram 1700 (FIG. 6). It should be appreciated that some of the operations of logic flow diagram 1700 can be performed by logic within survey taker device 1612 as described above. In operation 1702, generalized dialogue flow logic 302 (FIG. 3) can select a prompt or other dialogue action to initiate the dialogue with the survey taker.

Loop operation 1704 (FIG. 17) and next operation 1718 define a loop in which generalized dialogue flow logic 302 can conduct the behavioral health survey according to operations 1706-1716 until generalized dialogue flow logic 302 determines that the behavioral health survey is completed.

In operation 1706 (FIG. 17), generalized dialogue flow logic 302 (FIG. 3) can cause I/O logic 308 to carry out the dialogue action selected in operation 1702 or in the most recent performance of operation 1716 described below. If the selected dialogue behavior is to present the next prompt of the current behavioral health survey, generalized dialogue flow logic 302 can send prompt data representing the selected prompt to survey taker device 1612 (FIG. 16) so as to cause survey taker device 1612 to present the selected prompt to the survey taker.

In operation 1708, generalized dialogue flow logic 302 (FIG. 3) can receive response data representing the survey taker's response to the prompt and user interface metadata associated with the response data. In addition, survey annotation logic 202 can cause, through data access logic 306, survey annotation system data 206 to include the received response in individual responses 526 (FIG. 5), and the received user interface metadata in survey metadata 530, of survey record 520 representing the current, in-progress behavioral health survey.

In operation 1710 (FIG. 17), confidence annotation logic 304 (FIG. 3) can determine a degree of confidence in the reliability of the survey taker's response in a manner described above with respect to logic flow diagram 800 (FIG. 8). In operation 1712 (FIG. 17), confidence annotation logic 304 can update confidence vector 528 (FIG. 5) of survey record 520 representing the current, in-progress behavioral health survey such that confidence vector 528 represents an intermediate measure of confidence in the reliability of the survey taker's responses for the health survey so far.

In operation 1714 (FIG. 17), confidence annotation logic 304 can perform survey intervention analysis to determine whether intervention in the currently administered behavioral health survey is warranted. Using the updated, intermediate confidence vector determined in operation 1712, or a portion thereof, confidence annotation logic 304 can determine whether a dialogue action other than presenting the next prompt of the behavioral health survey is warranted. If the intermediate confidence vector is particularly low, confidence annotation logic 304 can determine that the next dialogue action is to terminate the behavioral health survey. If the intermediate confidence vector is somewhat low or portions of the intermediate confidence vector, particularly those pertaining to user interface metadata, indicate that the survey taker is rushing through the behavioral health survey without given careful consideration to the prompts of the survey, confidence annotation logic 304 can select a next dialogue action that is designed to slow the survey taker down and give more careful consideration to the prompts of the behavioral health survey. Examples of such dialogue actions can include, for example, asking the survey taker whether it is currently a convenient time to take the survey and even scheduling the survey taker to take the survey at a later time, prompting the survey taker to slow down, asking the survey taker to confirm a previously given response, causing the survey to be administered again as a multiple-pass behavioral health survey as described above, or pausing between presentation of survey prompts to the survey taker.

In operation 1716, generalized dialogue flow logic 302 (FIG. 3) can select the next dialogue action to be carried out in administration of the current behavioral health survey. Ordinarily, unless confidence annotation logic 304 determines that a particular dialogue action is to be taken next, the next dialogue action maybe the next prompt to be presented to the subject survey taker in the next performance of operation 1706 (FIG. 17). Processing transfers through next operation 1718 to loop operation 1704.

Generalized dialogue flow logic 302 (FIG. 3) can repeat the loop of operations 1704-1718 until generalized dialogue flow logic 302 determines that the behavioral health survey is complete and processing transfers from loop operation 1704 to operation 1720.

In operation 1720, confidence annotation logic 304 can determine a static confidence vector from the entirety of the results received in iterative performances of operation 1708 in the manner described above in conjunction with logic flow diagram 800 (FIG. 8) and combines the static confidence vector with the intermediate confidence vector resulting from operation 1712 (FIG. 17) and stores the result of the combination in confidence vector 528 (FIG. 5).

In test operation 1722 (FIG. 17), confidence annotation logic 304 can determine whether confidence vector 528 (FIG. 5) represents a measure of confidence that is below a predetermined threshold. If not, confidence annotation logic 304 can accept the results of the health survey as valid in operation 1724 and completes survey record 520 representing the current health survey by (i) recording the current date and time in time stamp 522 and (ii) determining and storing in score 524 a final score of the health survey in accordance with the received responses.

If confidence annotation logic 304 determines that confidence vector 528 (FIG. 5) represents a measure of confidence that is below the predetermined threshold in test operation 1722, confidence annotation logic 304 can reject the current health survey as invalid in operation 1726 (FIG. 17). In some embodiments, confidence annotation logic 304 rejects the current health survey as invalid by discarding survey record 520 representing the current health survey. In other, alternative embodiments, confidence annotation logic 304 may reject the current health survey as invalid by so marking survey record 520 representing the current health survey.

After operation 1724 or operation 1726, processing according to logic flow diagram 1700 completes. Thus, behavioral health survey confidence annotation machine 102 estimates a measure of confidence in the reliability of behavioral health survey results and can even terminate the behavioral health survey early upon determining that the confidence is below a predetermined threshold.

In some embodiments, survey annotation logic 202 can be implemented in survey taker device 1612 (FIG. 16) such that interactive administration of a behavioral health survey in the manner described in conjunction with logic flow diagram 1700 (FIG. 17) can be performed by survey taker device 1612 when offline, i.e., when not in communication with behavioral health survey confidence annotation machine 102. As described above, probability logic 408, cross-survey correlation logic 410, and metadata analysis can be simplified and only periodically updated such that survey annotation logic 202 can be implemented in a device with significantly fewer processing resources than behavioral health survey confidence annotation machine 102, e.g., survey taker device 1612.

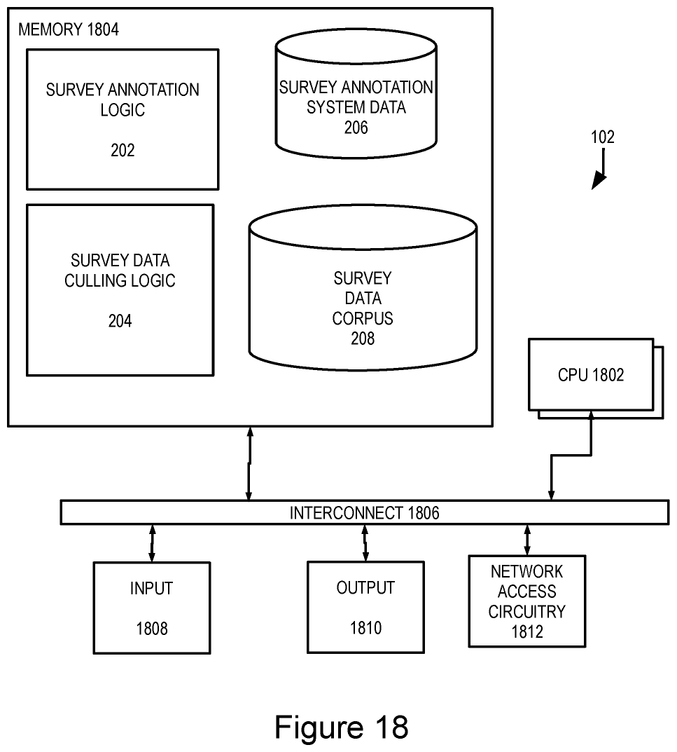

Behavioral health survey confidence annotation machine 102 is shown in greater detail in FIG. 18. As noted above, it should be appreciated that the behavior of behavioral health survey confidence annotation machine 102 described herein can be distributed across multiple computer systems using conventional distributed processing techniques. Behavioral health survey confidence annotation machine 102 may include one or more microprocessors 1802 (collectively referred to as CPU 1802) that retrieve data and/or instructions from memory 1804 and execute the retrieved instructions in a conventional manner. Memory 1804 can include generally any computer-readable medium including, for example, persistent memory such as magnetic and/or optical disks, ROM, and PROM and volatile memory such as RAM.

CPU 1802 and memory 1804 can be connected to one another through an interconnect 1806, which is a bus in this illustrative embodiment, and which connects CPU 1802 and memory 1804 to one or more input devices 1808, output devices 1810, and network access circuitry 1812. Input devices 1808 can generate signal in response to physical manipulation by a human user and can include, for example, a keyboard, a keypad, a touch-sensitive screen, a mouse, a microphone, and one or more cameras. Output devices 1810 can include, for example, a display—such as a liquid crystal display (LCD)—and one or more loudspeakers. Network access circuitry 1812 can send and receive data through computer networks such as WAN 1610 (FIG. 16). Server computer systems often exclude input and output devices, relying instead on human user interaction through network access circuity. Accordingly, in some embodiments, behavioral health survey confidence annotation machine 102 does not include input devices 1808 and output devices 1810.

A number of components of behavioral health survey confidence annotation machine 102 can be stored in memory 1804. In particular, survey annotation logic 202 and survey data culling logic 204 are each all or part of one or more computer processes executing within CPU 1802 from memory 1804 As used herein, “logic” refers to (i) logic implemented as computer instructions and/or data within one or more computer processes and/or (ii) logic implemented in electronic circuitry.

Survey annotation system data 206 and survey data corpus 208 are each data stored persistently in memory 1804 and can be implemented as all or part of one or more databases.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for measuring a degree of confidence in a reliability of responses received from a human subject in a health survey for evaluating a health state of the subject, the method comprising:
(a) obtaining response data that is generated by the subject in response to prompts presented to the subject during administration of the health survey to the subject, wherein the response data comprises a plurality of conditioning events and a plurality of conditioned events;
(b) determining a first probability that a first conditioned event is present in the response data based in part on a presence of a first conditioning event in the response data, wherein the plurality of conditioning events comprises the first conditioning event and the plurality of conditioned events comprises the first conditioned event, and wherein the first probability is used to determine a first event pair comprising the first conditioned event and the first conditioning event;
(c) repeating steps (a) and (b) for two or more other conditioned events and other conditioning events to generate a plurality of additional probabilities for a plurality of additional event pairs;
(d) combining two or more probabilities selected from (i) the first probability and the plurality of additional probabilities or (ii) the plurality of additional probabilities to generate a confidence vector data, wherein the confidence vector data represents a measure of confidence in the reliability of the subject that generated the response data in response to the health survey.
